(19) United States
(12) Patent Application Publication
(10) Pub. No.: US 2003/0064103 A1
(43) Pub. Date: Apr. 3, 2003

(54) Compositions and methods for treating vulvovaginitis and vaginosis

(76) Inventors: Shun Y. Lin, Plainsboro, NJ (US); Lorraine L. Wearley, Westfield, NJ (US); Henok Tassew, Highland Park, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/109,097

(22) Filed: Mar. 28, 2002

Related U.S. Application Data
(60) Provisional application No. 60/287,942, filed on May 1, 2001.

(51) Int. Cl. A61K 31/496; A61K 9/14

(52) U.S. Cl. 424/486; 514/254.07

(57) ABSTRACT

This invention relates to compositions and methods for treating vulvovaginitis and vaginosis. The compositions of this invention contain antifungal agents as well as a buffering system that, when administered to a patient's vagina, maintains the pH of the vagina so as to achieve a healthy environment that encourages the growth of appropriate flora. Antifungal agents that are useful in the compositions of this invention include azole antifungal agents. Buffering systems include gluconodeltalactone.

This application is a nonprovisional application based upon Provisional Patent Application No. 60/287,942 filed May 1, 2001.

### FIELD OF THE INVENTION

This invention relates to compositions and methods for treating vulvovaginitis and vaginosis utilizing antifungal agents in a buffered, pharmaceutically acceptable composition. Such antifungal agents may be applied to the vagina and vulvar area or intravaginally by sufferers of vulvovaginitis to relieve symptoms and effect treatment of vulvovaginitis, vaginal candidiasis and/or bacterial vaginosis at optimal conditions.

### BACKGROUND OF THE INVENTION

Bacterial vaginosis is a change in flora, the cause of which is still unknown in the vast majority of instances. Bacterial vaginosis has generally been used to represent any change in vaginal flora resulting in an assumed loss of lactobacilli. However, whether such flora represents the genetically normal state of the vagina in all women is poorly defined. Most therapies recommended for bacterial vaginosis in non-pregnant women are often successful in the short term, but usually unsuccessful if long-term follow-up is conducted. Although bacterial vaginosis is generally believed to be an endogenous condition, a number of behavioral factors are possibly involved, such as the use of contraceptive and intimate hygiene products and lifestyle habits. Although bacterial vaginosis is not considered a true sexually transmitted infection, it may be correlated to multiple sexual partners. Therefore, there is an increasing need to develop a product that is effective against bacterial vaginosis and other vaginitis.

Often, sufferers mistakenly think their vaginal infection is some type of a fungal infection such as Candida albicans that can be treated with over-the-counter (OTC) antifungal products. However, these OTC antifungal products are not effective treatments for bacterial vaginosis, a chronic condition which is estimated to be much more common than candidiasis.

Azole antifungal agents that are effective against vulvovaginitis caused by Candida albicans, and are available over the counter are good candidates to be formulate into products for bacterial vaginosis treatment, especially compounds such as miconazole that has activity in the vaginal pH environment. By increasing the effectiveness of selected azole compound(s) and conditioning the environment to alleviate microorganism growth. The invention describes formulations containing a buffered miconazole nitrate system for treating VVC, bacterial vaginosis, or VVC/bacterial vaginosis mixed infection.

Under stable conditions, the lactobacilli, the predominant organism in the normal vagina, control the growth of anaerobes and other bacteria by producing hydrogen peroxide and lactic acid from vaginal glycogen to maintain vaginal acidity. A vaginal pH between 4 and 5 is considered normal for women with active menstrual cycles. Bacterial vaginosis is one of the most common infectious disorders affecting women, accounting for 45% of symptomatic cases and estimated to be present in 15% of asymptomatic sexually active women.

Clinically, bacterial vaginosis is a polymicrobial vaginal infection caused by an increase in the number of anaerobic organisms with a concomitant decrease in lactobacilli in the vagina. Diagnosis of bacterial vaginosis is based on some noticeable symptoms: i) a thin, homogeneous discharge, ii) an elevated vaginal pH (>4.5), iii) a fishy odor from vaginal discharge and iii) some specific criteria: a) a 10% potassium hydroxide (amine) test, b) existence of clue cells. Currently, only two compounds have been used to treat bacterial vaginosis locally, products with either Metronidazole MetroGel-Vaginal®) or Clindamycin (Cleocin®). These two compounds are classified as antibacterial agents and Metronidazole is an anti-protozoal agent as well. They are available only by prescription. Because bacterial vaginosis is not a life threatening disease and the self diagnosis is not clearly defined, consumers have two options to treat their bacterial vaginosis infection: i) wait for days, even weeks, to seek a doctor's diagnosis, or ii) try to self-treat the infection with OTC products.

The probability of misdiagnosis by women who self-diagnose or even by physicians is fairly high. Although self-treatment with an OTC antifungal product generally cures VVC, where women have mixed infections, they may be likely to continue to have symptoms. Moreover, such coexisting infections require clinical and laboratory evaluation as well as treatment which addresses all aspects of the disease conditions.

Furthermore, studies have found that about 15-30% of patients who contract BV develop a post-treatment VVC infection due to the change in normal vaginal flora. Thus, having a single product or treatment regimen that can address both bacterial vaginosis and prevent subsequent fungal overgrowth would be extremely desirable.

In U.S. Pat. No. 5,536,743, for example, a buffered metronidazole-containing composition is described. However, this composition treats bacterial vaginosis only, as metronidazole is only effective against bacteria and not against fungi.

Vaginal pH is known to be an important factor in maintaining a healthy vaginal ecosystem. A study has been conducted by Milani et.al., Mipharm S.p.A., Milan, Italy to compare the effects of polycarbophil, a bioadhesive polymer in gel form, with those of an acidic vaginal douche, on restoration of physiologic vaginal pH in women whose vaginal pH was greater than 4.5 and who were suspected to have bacterial vaginosis. Both physical and microbiologic signs of bacterial vaginosis were improved in the polycarbophil group. The polycarbophil vaginal gel appears to reduce elevated vaginal pH to physiologic levels for 80 hours compared with acidic vaginal douche and to reduce vaginal pH in women with suspected bacterial vaginosis. However, this study did not address any means for treating a mixed infection.

Products sold in Mexico for treating both VVC and BV contain two active ingredients, such as itraconazole and secnidazole and. Such products thus contain one active ingredient for treating the fungal infection and one active ingredient for treating the bacterial infection. Combination products may be insufficient to treat sequential infections where either the fungal infection follows the bacterial infection or where the fungal component of a mixed infection is unmasked by treatment of a vaginal infection. Such a treatment also requires two active ingredients in one product in order to treat both infections.

Therefore, there exists a need for an over-the-counter product that is effective at treating both vulvovaginitis and bacterial vaginosis together or in sequence.

### SUMMARY OF THE INVENTION

The compositions and methods of this invention relate to products containing an antifungal compound and an active buffering compound as well as a pharmaceutically acceptable carrier. The buffered compositions of this invention are expected to have surprising effectiveness in treating both mycotic infections and bacterial vaginosis. The pH of the compositions of this invention are preferably maintained between about 2.5 and about 5.5. More preferably, the pH should be maintained between about 3 and about 5, most preferably between about 3 and about 4.5. At this pH range, both the antifungal compounds and the vaginal environment are conducive to treatment and prophylaxis of mycotic and bacterial vaginosis infections.

Thus, the compositions and methods of this invention relate to:

A composition for treating vulvovaginitis and vaginosis comprising:

a) an antifungal agent; and

b) a buffering system More preferably, this invention relates to a composition for treating vulvovaginitis and vaginosis comprising:

a) an azole antifungal agent; and

b) a buffering system comprising gluconodeltalactone. This invention also relates to a composition for treating vulvovaginitis and vaginosis comprising:

a) an azole antifungal agent;

b) a buffering system;

c) a pharmaceutically acceptable carrier. The compositions of this invention relate to an emulsion composition for treating vulvovaginitis and vaginosis comprising:

a) an azole antifungal agent;

b) a buffering system comprising gluconodeltalactone;

c) a carbomer; and

d) a pharmaceutically acceptable carrier; as well as a gel composition for treating vulvovaginitis and vaginosis comprising:

a) an azole antifungal agent;

b) a buffering system;

c) polyethylene glycol; and

d) a pharmaceutically acceptable carrier. The compositions and methods of this invention also relate to a dual-phase composition for treating vulvovaginitis and vaginosis comprising:

a) an oil phase comprising an azole antifungal agent and oil; and

b) a water phase comprising a buffering system and water; and

c) a pharmaceutically acceptable carrier. The methods of this invention relate to a method for treating vulvovaginitis and vaginosis comprising administering to a vaginal mucous membrane a composition comprising an azole antifungal agent and a buffering system. The compositions and methods of this invention may also be useful in preventing, i.e., in the prophylaxis, of vaginal infections in accordance with the compositions and methods set forth above. The compositions of this invention may also be packaged in a kit containing the compositions according to this invention as well as a soothing composition containing anti-irritant, anti-inflammatory, emollients, antifungal, antiseptic and like ingredients which can be applied to the vulvar skin in order to soothe and protect the skin and help it to heal.

Surprisingly, although miconazole nitrate is not generally effective against bacterial infections, we have found that its antibacterial activity is significantly enhanced by buffering.

### DETAILED DESCRIPTION OF THE PREFERRED EMBODIMENTS

Vaginal infections such as candidiasis-related infection require an active antifungal compound in the dosage form to treat the infection. Azole-type antifungals are known for effectiveness in treating vaginal mycotic infections without disrupting the vaginal flora. Several azole compounds with proven efficacy against fungal infection have been approved for OTC use, including vaginal products containing miconazole nitrate, tioconazole, or clotrimazole. Therefore, the safety of these azole products has been established.

Although the effectiveness of these VVC effective azole products for treating bacterial vaginosis related infections has not been proven, using the compositions of this invention, there exists an opportunity to develop an effective dosage from these safe antimycotic-effective compounds for vaginal infections such as candidiasis, bacterial vaginosis, and mixed infections.

The novel compositions of this invention, which combine the antimycotic effectiveness of antifungal ingredients with a buffered carrier composition, maintain or adjust the vaginal pH to healthy levels and permit treatment and, potentially, prophylaxis, of both vulvovaginitis and bacterial vaginosis.

The dose of antifungal agent for treating vulvovaginitis and bacterial vaginosis varies depending upon the antifungal active ingredient used and its potency. The amount of the antifungal ingredient effective to treat an infection is referred to as the “therapeutically effective amount”. The antifungal agent in the compositions of this invention should preferably be present in a therapeutically effective amount. Preferably, they should be present in an amount from about 0.01% to about 90% weight by weight of the composition. More preferably, they should be present in an amount from about 0.1% to about 50% weight by weight, more preferably in an amount from about 0.4% to about 10% weight by weight. The buffering agent in the composition should be present in an amount of from about 0.01% to about 50% w/w. More preferably, it should be present in an amount of from about 0.1% to about 20% w/w and most preferably, from about 1% to about 5% weight by weight.

Other components may be present in the compositions of this invention such as water, anti-oxidants, chelating agents, preservatives, oils, waxes, surfactants, emulsifiers, viscosity building agents, solvents, moisturizing agents, solubilizers and bioadhesives/muco-adhesives and the like. The relative quantities of such components may vary according to the desired nature and consistency of the composition, including creams, ointments, waxy suppositories, gelatin capsules, anhydrous polymeric suppositories and the like.

The preferred buffered forms of the compositions of this invention may be made as emulsions, gels or as two-phase, or dual, dosage forms. Preferably, one hydrophilic phase is present in the compositions of the invention in order to provide a sector of the composition, which can be buffered. Three preferred buffered dosage form designs containing an active antifungal compound against are as follows: i) A hydrophilic cream, ii) hydrophilic gel, iii) and a two-phase dosage form design for treating vaginal infections described above. These would ease consumers' desire for immediate and effective treatment of vaginal infection. The buffer capacity of each formulation is formulated to be able to maintain the pH at a level of from about 3 to about 5.5, more preferably from about 3 to about 4.5.

The buffering agents according to this invention may be applied into the vagina prior to, during, or after an intravaginal antifungal drug treatment. For example, the buffering agents may be incorporated directly into a composition containing an antifungal azole compound. In this case, the buffering agent and the azole compound are preferably administered to patients simultaneously during application. The dosing regimen will vary depending upon the particular antifungal agent that is being employed in the products of the invention. A therapeutically effective or prophylactically effective dose should be employed.

Alternatively, the buffering agent may be administered before and/or after the intravaginal antifungal treatment. For example, the buffering agents may be coated on the outer surface of an vaginal suppository (e.g., a wax- or fatty acid based antifungal vaginal suppository), or a gelatin capsule suppository containing an antifungal drug. The buffering agents can also be incorporated into the gelatin-wall of the antifungal gelatin capsule.

Preferably, buffering agents are incorporated into certain polymeric or biopolymer muco-adhesive materials, such as gelatin, chitosan and its derivatives, hydrophilic cellulose (preferably a hydroxyalkylcellulose and more preferably, hydroxymethylcellulose, hydroxyethylcellulose, or the like or a mixture thereof), and polyacrylate-polyacrylic acid polymers (e.g., Carbomers and the like). The hydrophilic polymer containing buffering agents may serve as gelling agent in a gel-type composition, or viscosity-building agent in an emulsion-type composition as in, for example, an oil-in-water cream.

Alternatively, the buffering agent-embedded hydrophilic polymer may be suspended in a lipophilic composition containing an antifungal drug (for example, an ointment, a wax-/fatty acid-suppository, or a water-in-oil emulsion). Upon application into the vagina, the hydrophilic polymer will adhere to the vaginal mucosal membrane, thus maintaining the vaginal pH at the preferable pH range for a prolonged period of time, even long after the antifungal drug has been eliminated or excreted from the vagina. Such prolonged maintenance of vaginal acidity assures re-establishment of healthy microbial flora (e.g., Lactobacillus species), and inhibits opportunistic pathogenic yeast (e.g., Candida albicans) in the vagina.

The compositions of this invention should contain at least one active antifungal ingredient, preferably an azole antifungal ingredient. More preferably, such compounds are miconazole nitrate, terconazole, butaconazole, itraconazole, voriconazole, ketoconazole, econazole, tioconazole, fluconazole, posconazole, ravuconazole, clotrimazole and the like.

The compositions of this invention should also contain at least one buffering system or agent. Preferably, such buffering agent is gluconodeltalactone (“GDL”). GDL is a neutral cyclic ester of gluconic acid. When added into an aqueous solution, GDL rapidly dissolves, and subsequently slowly hydrolyzes to gluconic acid. Other buffering systems or agents may be used as well in the compositions and methods of this invention. The term “buffer system” or “buffer” as used herein refers to a solute agent or agents which, when in aqueous solution, stabilize such solution against a major change in pH (or hydrogen ion concentration) when acids or bases are added thereto. Solute agent or agents which are used for a resistance to change in pH from a starting buffered pH value around pH 4 as preferably utilized in the compositions and methods of this invention. In general, buffers for the compositions of this invention include any physiologically acceptable organic acid and its corresponding salt, either liquid or solid no (depending upon the desired form of application. Preferably, such buffers have a pKa from about pH 3 to about pH 5. Buffers preferably useful in the compositions and methods of this invention include, but are not limited to, acetic, fumaric, lactic, citric, propionic, lactic, malic, succinic, gluconic, ascorbic, tartaric acids and the like. Polymers with ionizable functional groups, including, for example, a carboxylic acid or an amine group, and a buffering capacity may also be used as polymeric buffers according to this invention. Examples of polymeric buffers preferably used in the compositions and methods of this invention include Carbomer® or Carbopol®, available commercially from B.F. Goodrich Co., Akron, Ohio, and carboxymethyl celluloses. Virtually any pharmaceutically acceptable buffer system that achieve a pH in the preferred range for topical applications may be used in the compositions and methods of this invention.

Buffered formulations of an azole suitable for vaginal application according tot he present invention and suitable for achieving the desired therapeutic action and physiological pH of the vagina of about 4 may be formulated in any convenient non-flowing form, including, but not limited to, suspensions, emulsions, clear and opaque gels, semisolid systems, including ointments, pastes, oil-in-water (o/w) creams, semisolid emulsions with solid internal phases, semisolid emulsions with fluid internal phases, vaginal suppositories, insertable tablets, soft or hard gelatin capsules and the like.

Surprisingly, it was found that a buffered gel containing an azole antifungal agent, miconazole nitrate, had a better buffer capacity with a pH of between about 3 and about 5.5 than buffered gels that did not contain miconazole nitrate.

The compositions of this invention may also contain other ingredients for use in emulsified, gel or two-phase systems. For example, emulsions may contain surfactants, oils, humectants, pH adjustors, waxes, polymer carriers, bioadhesives and water known to those of ordinary skill in the art. Gel formulations may contain oils, humectants, carbomers, cellulose, polyalkylene glycols and water, in addition to the active ingredients and buffer systems. The compositions may be in the form of creams, suppositories, gels or dual-phase combinations.

The two-phase dosage form of this invention preferably contains a buffered gel and a hydrophobic antifungal component in a delivery system. The hydrophobic phase of the combination is stable inside the delivery system and is designed to melt at body temperature. Such a dosage form may be delivered, both phases together, by an applicator which is capable of insertion into the vaginal cavity. Advantageously, a two-phase dosage form permits simultaneous delivery of antifungal medication and buffering gel to the vagina, thus providing treatment capability of both mycotic and bacterial infections. The antifungal medication fights mycotic infections while the buffering gel lowers and maintains the pH of the vagina in a healthy range.

The method of using the compositions of this invention provides treatment for mycotic vulvovaginitis and bacterial vaginosis. The compositions are administered to the vaginal cavity by insertion therein. Preferably, a bioadhesive component within the compositions of this invention provides retention of the active ingredient and the buffering system in conjunction with the mucosal membranes of the vagina. The compositions may be reapplied daily until any abnormal flora, including fungus and/or bacteria, are destroyed and the infection is cured.

The following examples are merely illustrative of several of the possible compositions of this invention. The examples serve only to illustrate, and not to limit, the compositions and methods of this invention.

| Ingredient | % w/w (1A) | % w/w (1B) | % w/w (1C) | % w/w (1D) |
|---|---|---|---|---|
| Stearyl Alcohol | 8.500 | 8.500 | 8.500 | 8.500 |
| Cetyl Alcohol | 3.000 | 3.000 | 3.000 | 3.000 |
| Polysorbate 60 | 3.000 | 3.000 | 3.000 | 3.000 |
| Isopropyl Myristate | 1.000 | 1.000 | 1.000 | 1.000 |
| Propylene Glycol | 20.000 | 20.000 | 20.000 | 20.000 |
| Benzoic Acid | 0.200 | 0.200 | 0.200 | 0.200 |
| Potassium Hydroxide | 0.055 | 0.055 | 0.055 | 0.055 |
| Glucono Delta Lactone (GDL) | 1.000 | 1.000 | 1.800 | 0.900 |
| Carbomer (Carbopol 974P) | — | 2.000 | — | 0.900 |
| Miconazole Nitrate | 4.000 | 4.000 | 4.000 | 4.000 |
| Purified Water | 59.245 | 57.245 | 58.445 | 58.445 |

The composition of this example may be prepared using the following procedure:

1. Add water and propylene glycol to a container and heat to from about 70 to about 75° C. while mixing at low speed with paddle stirrer. When the mixture reaches the desired temperature(s), add benzoic acid with continuous mixing. When the benzoic acid is dissolved add potassium hydroxide and mix until dissolved.

2. When the potassium hydroxide is dissolved, add polysorbate 60 and mix for about 1 minute while maintaining the batch temperature at 70-75° C. Then stop the mixer and add isopropyl myristate, cetyl alcohol, and stearyl alcohol. Mix the batch again at from about 70 to about 75° C. until all ingredients in the container are completely dispersed.

3. Remove the container from the heat source and continue mixing using a homogenizer for about two minutes. After homogenization, mix the batch with paddle stirrer while cooling the batch to about 40° C.

4. When the temperature reaches about 40° C., add miconazole nitrate to the container with mixing. After adding miconazole nitrate, add glucono delta lactone to the container and homogenize the mixture for about four minutes or until the miconazole nitrate is completely dispersed. After homogenization, continue mixing with a paddle stirrer for about 5 minutes.

### EXAMPLE#2

### Buffered Placebo Cream

| Ingredient | % w/w (2A) | % w/w (2B) |
|---|---|---|
| Stearyl Alcohol | 8.500 | 8.500 |
| Cetyl Alcohol | 3.000 | 3.000 |
| Polysorbate 60 | 3.000 | 3.000 |
| Isopropyl Myristate | 1.000 | 1.000 |
| Propylene Glycol | 20.000 | 20.000 |
| Benzoic Acid | 0.200 | 0.200 |
| Potassium Hydroxide | 0.055 | 0.055 |
| Glucono Delta Lactone (GDL) | — | 1.800 |
| Carbomer (974) | — | — |
| Miconazole Nitrate | — | — |
| Purified Water | 64.245 | 62.445 |

The composition of this example may be prepared using the following procedure:

1. Add water and propylene glycol to a container and heat to from about 70 to about 75° C. while mixing at low speed with paddle stirrer. When the mixture reaches the desired temperature(s), add benzoic acid with continuous mixing. When the benzoic acid is dissolved add potassium hydroxide and mix until dissolved.

2. When the potassium hydroxide is dissolved, add polysorbate 60 and mix for about 1 minute while maintaining the batch temperature at 70-75° C. Then stop the mixer and add isopropyl myristate, cetyl alcohol, and stearyl alcohol. Mix the batch again at from about 70 to about 75° C. until all ingredients in the container are completely dispersed.

3. Remove the container from the heat source and continue mixing using a homogenizer for about two minutes. After homogenization, mix the batch with paddle stirrer while cooling the batch to about 40° C.

4. When the temperature reaches about 40° C. add glucono delta lactone if needed to the container and homogenize the mixture for about four minutes or until the miconazole nitrate is completely dispersed. After homogenization, continue mixing with a paddle stirrer for about 5 minutes.

### EXAMPLE#3

### Single-Carbomer Gels

| | | % w/w, with |
|---|---|---|
| | % w/w, | Azole compound |
| Ingredient | placebo (3A) | (3B) |
| Potassium Chloride | 0.16 | 0.16 |
| EDTA | 0.02 | 0.02 |
| Carbomer 974P (Carbopol 974P, | 2.08 | 2.08 |
| B.F. Goodrich) | | |
| Sodium Hydroxide | 0.17 | 0.17 |
| Miconazole Nitrate | — | 4.00 |
| Purified Water | 97.57 | 93.57 |

The composition of this example may be prepared using the following procedure:

1. Add Carbomer 974P into water and mix using a high speed mixer at room temperature, such as homogenizer

2. Then add potassium chloride, EDTA, and sodium hydroxide and mix using a low speed mixer, such as paddle mixer

3. For the formulation containing azole compound, add the miconazole nitrate into the mixture and mix using both homogenizer and paddle to have a uniform dispersion of miconazole nitrate in the formulation.

### EXAMPLE#3

### Single-Carbomer Gels

| | | % w/w, with |
|---|---|---|
| | % w/w, | Azole compound |
| Ingredient | placebo (3A) | (3B) |
| Potassium Chloride | 0.16 | 0.16 |
| EDTA | 0.02 | 0.02 |
| Carbomer 974 (Carbopol 974P, | 2.08 | 2.08 |
| B.F. Goodrich) | | |
| Sodium Hydroxide | 0.17 | 0.17 |
| Miconazole Nitrate | — | 4.00 |
| Purified Water | 97.57 | 93.57 |

The composition of this example may be prepared using the following procedure:

4. Add Carbomer 974P into water and mix using a high speed mixer at room temperature, such as homogenizer

5. Then add potassium chloride, EDTA, and sodium hydroxide and mix using a low speed mixer, such as paddle mixer

6. For the formulation containing azole compound, add the miconazole nitrate into the mixture and mix using both homogenizer and paddle to have a uniform dispersion of miconazole nitrate in the formulation.

### EXAMPLE#4

### Multi-Carbomer Gels

| | | % w/w | | % w/w |
|---|---|---|---|---|
| | % w/w | placebo, | % w/w | placebo, |
| Ingredient | (4A) | (4B) | (4C) | (4D) |
| Carbomer 971 | 2.00 | 2.00 | 2.00 | 2.00 |
| Mineral Oil | 4.20 | 4.20 | 4.20 | 4.20 |
| Glycerin | 12.90 | 12.90 | — | — |
| Carbomer 974 | 1.00 | 1.00 | 1.00 | 1.00 |
| Distilled monoglycerides | 1.00 | 1.00 | 1.00 | 1.00 |
| Sorbic Acid | 0.08 | 0.08 | 0.08 | 0.08 |
| Polyethylene Glycol 400 | — | — | 12.90 | 12.90 |
| Miconazole Nitrate | 4.00 | — | 4.00 | — |
| Purified Water | 74.82 | 78.82 | 74.82 | 78.82 |

The composition of this example may be prepared using the following procedure:

1. Add glycerin, mineral oil (or polyethylene glycol 400), distilled monoglycerides (such as Myverol), and sorbic acid into a suitable container and heat to 65-70° C. Then add Carbomer 971 and 974 into the container and mix.

2. Heat the water separately to 55-60° C. and then add to the mixture from (1). Mix for about 3 minutes before adding miconazole nitrate into the container.

3. Mix the batch with a paddle stirrer while cooling down to about 45° C. When the temperature about 45° C., mix the batch using a homogenizer for about 2 minutes.

4. Switch the mixing method back to the paddle stirrer while cooling the batch to room temperature.

### EXAMPLE#5

### Carboxymethylcellulose Gels

| | % w/w | % w/w (placebo) |
|---|---|---|
| Ingredient | (5A) | (5B) |
| Glucono Delta Lactone (GDL) | 2.50 | 2.50 |
| Sodium Hydroxide | 0.25 | 0.25 |
| Methylparaben | 0.20 | 0.20 |
| Glycerin | 17.00 | 17.00 |
| Hydroxyethylcellulose | 3.00 | 3.00 |
| Miconazole Nitrate | 4.00 | — |
| Purified Water | 73.05 | 77.05 |

The composition of this example may be prepared using the following procedure:

1. Add Hydroxyethylcellulose into water and mix using a high speed mixer at room temperature, such as homogenizer

2. Then add glycerin, methylparaben, sodium hydroxide, and glucono delta lactone and mix using a low speed mixer, such as paddle mixer

3. Add the miconazole nitrate into the mixture and mix using both homogenizer and paddle to have a uniform dispersion of miconazole nitrate in the formulation.

### EXAMPLE#6

### Adhesive/Hydrophobic Suppository

| Ingredient | % w/w (6A) | % w/w (6B) |
|---|---|---|
| Xanthan Gum | 1.00 | 1.00 |
| Sodium Carboxymethylcellulose 7HF | 8.00 | 8.00 |
| Colloidal Silicon Dioxide | 1.00 | 1.00 |
| Wecobee M | 12.00 | 15.00 |
| Wecobee FS | 54.00 | 67.00 |
| Miconazole Nitrate | 24.00 | 8.00 |

The composition of this example may be prepared using the following procedure:

1. Melt the Wecobee M and FS, (which are hard fat bases consisting primarily of mixtures of the triglyceride esters of the higher saturated fatty acids along with varying proportions of mono- and diglycerides) in a suitable container at 50 to 60° C. Add xanthan gum, colloidal silicon dioxide, and sodium carboxymethylcellulose 7HF into the container with proper mixing. Continue mixing with a homogenizer for about 2 minutes or until the additives are fully dispersed.

2. Add the miconazole nitrate into the batch while mixing with a homogenizer. Cool the batch to room temperature while mixing with a low speed mixer. The batch solidifies at temperature <35° C.

### EXAMPLE #7

### Buffering Capacity

In order to determine the buffering capacity of the compositions of this invention, the following procedure was used.

The amounts of 0.1N sodium hydroxide to change the pH of samples described in Examples 1-5 were determined by a titration method. The amount of sodium hydroxide solution added to the samples in molar-equivalent basis is presented in the following graphs.

The sample produced from Example 6 contained no buffer capacity between 3.0 and 5.5 and is designed to be delivered with a placebo buffering gel (Example 5) in an applicator. This is an example of the described two-phase delivery system. Data obtained for Buffered Metrogel-Vaginal® treatment (available from 3M Corporation, Minneapolis, Minn.)for bacterial vaginosis treatment is provided for comparison as set forth as the comparator in the Figures.

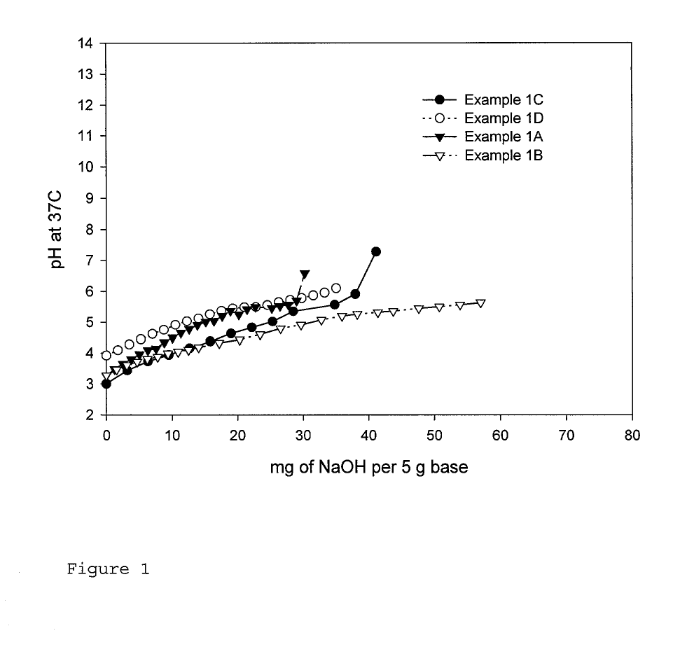

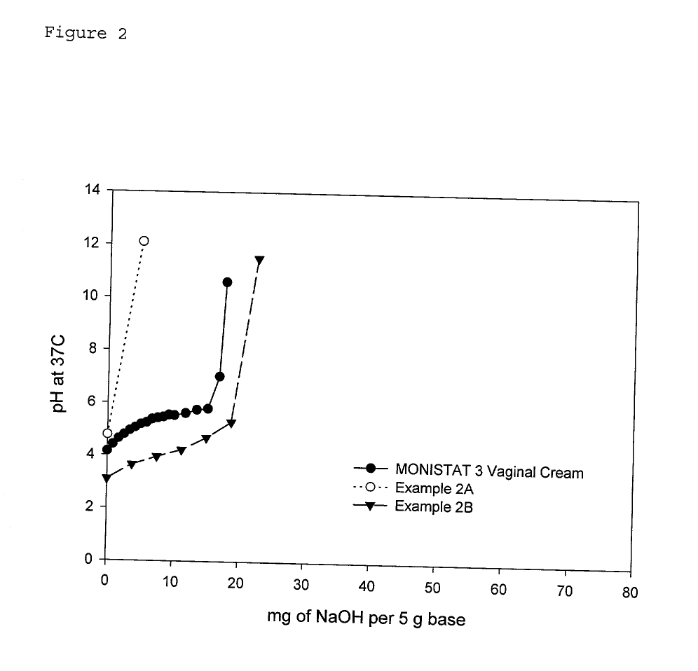

FIG. 1 and demonstrate the buffer capacity for the aforementioned cream formulations of Examples 1 and 2. Monistat 3® vaginal cream is used as a control. As shown, Examples 1A, 1B, 1C, 1D and 2B have relatively good buffering capacity while comparative Example 2A and Monistat 3® vaginal cream do not. Example 2A does not contain either buffer or carbomer. The buffer capacity of cream base is improved significantly after addition of 1.8% or more of glucono delta lactone or a combination of gluconodelta lactone and carbomer. A better buffer capacity is also observed for formulations containing miconazole nitrate as compared with placebo (Example 1C as compared with Example 2B). This is surprising indicating that the miconazole nitrate could enhance the buffer capacity in the described cream formulations.

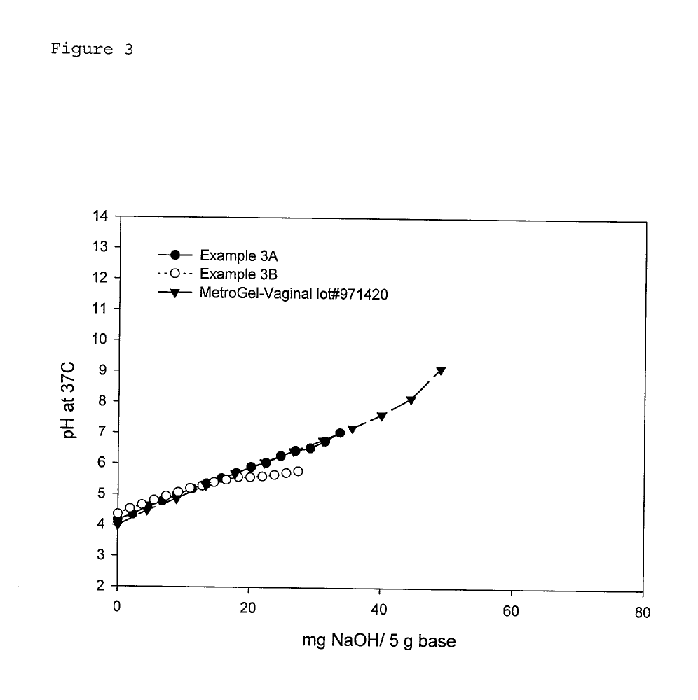

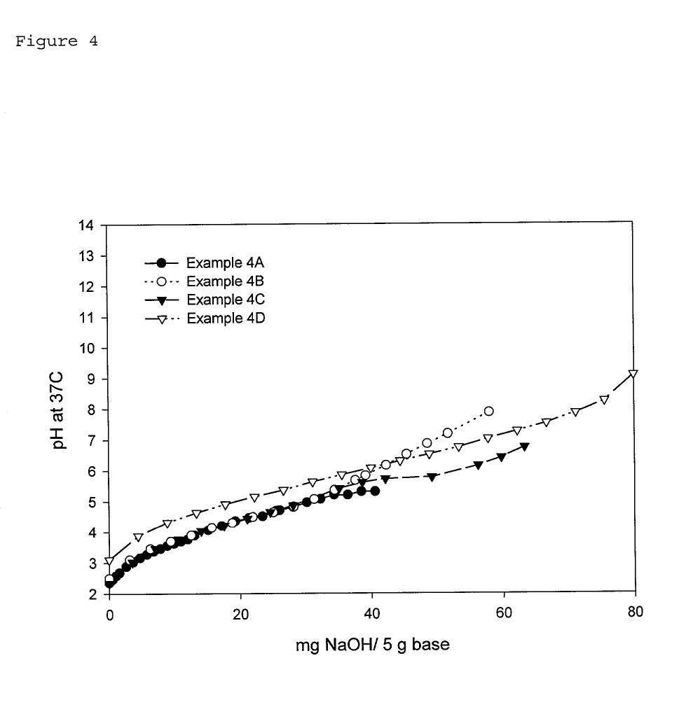

FIGS. 3 and 4 demonstrate buffered gel formulations of Examples 3 and 4, compared to MetroGel-Vaginal®, a commercial formulation for treating bacterial vaginosis. Formulations 3A, 4B and 4D do not contain miconazole nitrate and have relatively less buffering capacity than the other formulations containing miconazole nitrate (3B, 4A, and 4C).

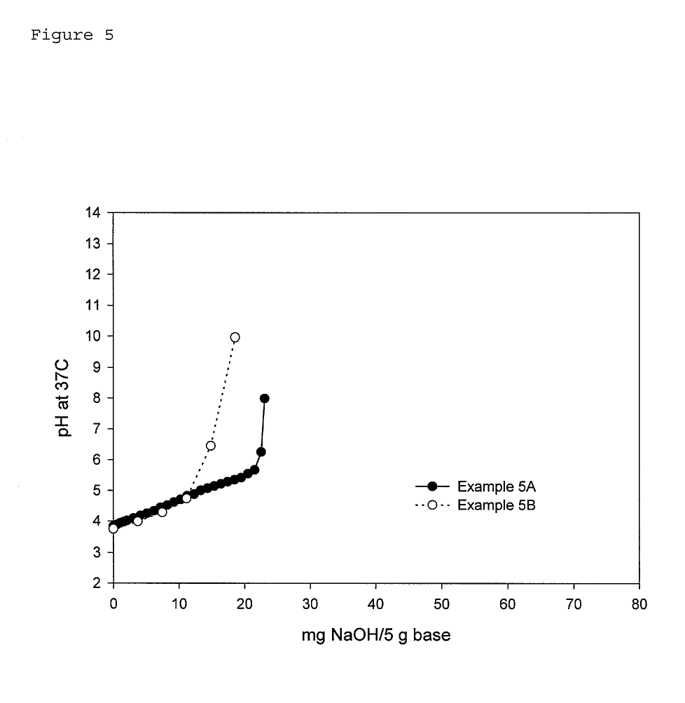

FIG. 5 demonstrates buffered gel formulations of Example 5. Formulation 5B does not contain miconazole nitrate and have relatively less buffering capacity than the other examples.

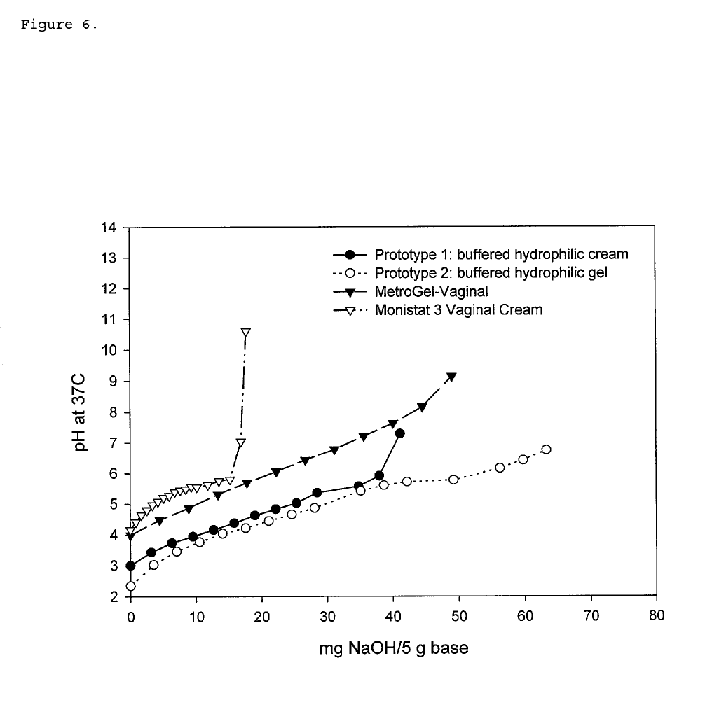

FIG. 6 demonstrates a comparison between preferred buffered formulations of this invention, formulations 1C and 4C, compared to MetroGel-Vaginal®, a commercial formulation containing metronidazole for treating bacterial vaginosis locally and Monistat 3® Vaginal Cream, a commercial formulation containing miconazole nitrate for treating vulvovaginal candidiasis locally. As demonstrated, the compositions of this invention are more capable of maintaining a healthy pH by buffering capacity than the commercial products.

### EXAMPLE 8

### In Vitro Evaluation of Antibacterial Vaginosis Organism Activities

The ability of selected vaginosis anaerobes to survive in a mixture of disclosed formulations and supplemented brucella broth was also studied. The brucella broth, supplemented with vitamin K and hemin, was prepared in double strength to allow for dilution with the formulations of this invention. Studied organisms were taken from a freezer and sub-cultured at least twice to ensure purity and good growth. The following procedures were used to perform the in vitro evaluation:

Method: Steer's Replicator Assay (survival time in hrs)

1. Mix the test sample 1 gram plus 9 ml dimethyl sulphoxide (“DMSO”). One of the preparations should be melted at 40-46° C. and mixed thoroughly prior to dissolving in DMSO. Prepare 18 ml. Remove a small quantity and measure and record the pH. Pass into chamber and allow to become anaerobic for at least 2 hrs.

2. Working in the chamber, prepare a suspension equal to the #1 McFarland equivalence turbidity standard for each anaerobic organism in double strength-supplemented brucella broth (˜3×10⁸ cfu/ml). Add 0.5 ml to the steers replicator wells. Stamp one BBA (brucella blood agar) plate as a pre-growth control.

3. Add 0.5 ml of the cream solution to the broth to each of the wells, using multi-channel pipettor. Mix thoroughly by pipetting up and down. When completed, record how long it took to inoculate the entire replicator head. (first wells will have had a longer contact time than last wells). Stamp a BBA plate as “0” time. Use one steer's replicator head for each of the creams. Each day of the test set-up, prepare a control replicator with organisms' suspensions plus brucella broth and DMSO (1+9), but no cream.

4. Incubate with prongs in the wells.

5. Every hour, stamp another BBA and label the plate with the time in hours. Incubate at 36° C.

6. Continue to 24 hours.

7. Examine the stamped plates after 72 hours of incubation and record if there is growth or no growth or describe type of growth i.e. few colonies, hazy growth etc. that might suggest damaged cells.

8. Final report is reported in the time in hours that the organism survived in the presence of each of the samples in Table I below.

For the azole compounds studied, miconazole, terconazole, and fluconazole are approved azoles for treating vulvovaginal candidiasis. Metronidazole and tinidazole are compounds known to be useful for treating bacterial vaginosis. However, the unexpected finding from this in vitro evaluation of azole compounds is that the miconazole actually has a better activity against bacterial vaginosis organisms than terconazole and fluconazole.

TABLE I
Results of In Vitro Evaluation: Activities of Azole compounds
Present range of MICs for each organism instead of individual rows

| | MIC's (μg/ml) of drug needed to inhibit the growth of organism | | | | |
|---|---|---|---|---|---|
| Organism | Metronidazole | Miconazole | Tinidazole | Terconazole | Fluconazole |
| Gardnerella vaginalis | 8 | 16 | >128 | 64 | >2048 |
| Gardnerella vaginalis | 4 | 32 | 1 | 64 | >2048 |
| Gardnerella vaginalis | >32 | 16 | 128 | 64 | >2048 |
| Gardnerella vaginalis | >32 | >128 | 16 | 256 | >2048 |
| Peptostreptococcus magnus | 0.5 | 64 | 0.25 | 256 | >2048 |
| Peptostreptococcus magnus | 1 | 32 | 0.5 | 256 | >2048 |
| Peptostreptococcus magnus | 0.25 | >128 | 0.125 | 256 | >2048 |
| Peptostreptococcus tetradius | 1 | 128 | 0.5 | 256 | >2048 |
| Peptostreptococcus tetradius | 1 | 128 | 0.5 | 256 | >2048 |
| Peptostreptococcus tetradius | 0.5 | 16 | 0.25 | No growth | 2048 |
| Peptostreptococcus | 2 | 64 | 1 | 256 | >2048 |
| asaccharolyticus | | | | | |
| Peptostreptococcus | 0.25 | 16 | 1 | 256 | >2048 |
| asaccharolyticus | | | | | |
| Peptostreptococcus | 0.5 | 64 | 1 | 256 | >2048 |
| asaccharolyticus | | | | | |
| Prevotella bivia | 1 | 128 | 1 | 256 | >2048 |
| Prevotella bivia | 1 | 64 | 1 | 256 | >2048 |
| Prevotella disiens | 0.5 | 64 | 0.125 | No growth | >2048 |
| Prevotella disiens | 0.5 | 64 | 1 | 128 | >2048 |
| Prevotella disiens | 1 | 64 | 1 | 256 | >2048 |
| Prevotella intermedia | 1 | 64 | 0.5 | 128 | >2048 |
| Prevotella intermedia | 1 | 64 | 1 | 256 | >2048 |
| Prevotella melaninogenica | 1 | 64 | 2 | 64 | >2048 |
| Prevotella melaninogenica | 0.25 | 64 | 1 | No growth | >2048 |
| Mobiluncus mulieris | 4 | 8 | 1 | 256 | >2048 |
| Bacillus fragilis | 0.5 | >128 | 0.5 | 256 | >2048 |
| Bacillus theta | 2 | 128 | 1 | 256 | >2048 |
| Lactobacillus plantarum | 1 | 32 | 0.65 | 256 | >2048 |
| Lactobacillus species | >32 | >128 | >128 | 512 | >2048 |
| Lactobacillus acidophilus | >32 | >128 | >128 | 512 | >2048 |
| Lactobacillus acidophilus | >32 | >128 | <128 | 512 | >2048 |

The activity of disclosed formulations against bacterial vaginosis organisms are shown in the following Table II. Among the formulations studied, the examples 2A, 2B, 4B, and 4D are formulations without miconazole nitrate. The example 2A which has the lowest buffer capacity, shows the least effectiveness against the studied organisms. The example 2B is the buffered placebo formulation of example 1C and the example 4D is the buffered placebo formulation of example 4C. The activity is against the studied organisms is enhanced significantly by incorporating the miconazole nitrate into the example 1C. Same results are obtained by incorporating the miconazole nitrate into the example 4D.

TABLE II
Results of In Vitro Evaluation: Activities of Formulations of the Invention

| | EXAMPLE | | | | | | | | |
|---|---|---|---|---|---|---|---|---|---|
| | | | | | Monistat 3 | | | | |
| | | | | | vaginal | | | | MetroGel- |
| Organism | 4B | 5B | 4C | 4D | cream | 1C | 2A | 2B | Vaginal |
| Gardnerella vaginalis | 2 | >7 < 24 | 0 | 1 | 0 | 0 | 0 | 1 | 2 |
| Gardnerella vaginalis | 1 | >7 < 24 | 0 | 3 | 0 | 0 | 0 | 2 | 4 |
| Gardnerella vaginalis | 1 | >7 < 24 | 0 | 3 | 0 | 0 | 0 | 4 | >9 < 23 |
| Gardnerella vaginalis | 3 | >7 < 24 | 1 | 7 | 2 | 1 | 6 | >24 | >9 < 23 |
| Peptostreptococcus magnus | 4 | >7 < 24 | 1 | 7 | 6 | 1 | >24 | 23 | 0 |
| Peptostreptococcus magnus | 4 | >7 < 24 | 1 | 6 | 5 | 1 | >24 | >24 | 0 |
| Peptostreptococcus magnus | 2 | >7 < 24 | 1 | 3 | 4 | 1 | >24 | >24 | 0 |
| Peptostreptococcus tetradius | 1 | >7 < 24 | 0 | 1 | 1 | 1 | >24 | >9 < 23 | 0 |
| Peptostreptococcus tetradius | 0 | >7 < 24 | 0 | 5 | 1 | 1 | >24 | >9 < 23 | 0 |
| Peptostreptococcus tetradius | 1 | >7 < 24 | 1 | 5 | 2 | 0 | >24 | >9 < 23 | 0 |
| Peptostreptococcus | 2 | >7 < 24 | 1 | 3 | 2 | 1 | >24 | >9 < 23 | 0 |
| asaccharolyticus | | | | | | | | | |
| Peptostreptococcus | 2 | >7 < 24 | 1 | 3 | 2 | 1 | >24 | 23 | 0 |
| asaccharolyticus | | | | | | | | | |
| Peptostreptococcus | 2 | >7 < 24 | 1 | 5 | 1 | 1 | >24 | >9 < 23 | 0 |
| asaccharolyticus | | | | | | | | | |
| Prevotella bivia | 2 | >7 < 24 | 1 | 4 | 1 | 1 | >24 | >9 < 23 | 0 |
| Prevotella bivia | 2 | >7 < 24 | 1 | 4 | 1 | 1 | >24 | >24 | 0 |
| Prevotella bivia | 2 | 6 | 1 | 8 | 1 | 1 | >24 | >9 < 23 | 0 |
| Prevotella disiens | 1 | >7 < 24 | 0 | 2 | 1 | 1 | >24 | 7 | 0 |
| Prevotella disiens | 1 | >7 < 24 | 0 | >24 | 1 | 0 | >24 | 4 | 0 |
| Prevotella disiens | 1 | >7 < 24 | 1 | >24 | 1 | 1 | >24 | 8 | 0 |
| Prevotella intermedia | 0 | 4 | 0 | >8 < 23 | 1 | 1 | >24 | 7 | 0 |
| Prevotella intermedia | 0 | 3 | 0 | >8 < 23 | 1 | 1 | >24 | 7 | 0 |
| Prevotella melaninogenica | 1 | 6 | 1 | >8 < 23 | 1 | 1 | >24 | >9 < 23 | 0 |
| Prevotella melaninogenica | 1 | >7 < 24 | 0 | 1 | 1 | 1 | >24 | >9 < 23 | 0 |
| Mobiluncus mulieris | 24 | 24 | >24 | 1 | >24 | 23 | 1 | 1 | 1 |
| Bacillus fragilis | >7 < 24 | 24 | 1 | >24 | 3 | 1 | >24 | >24 | 0 |
| Bacillus theta | >7 < 24 | 24 | 1 | >8 < 23 | 2 | 1 | >24 | >24 | 0 |
| Lactobacillus plantarum | 1 | >7 < 24 | 1 | 5 | 1 | 1 | >24 | 2 | 0 |
| Lactobacillus species | >7 < 24 | 24 | 5 | >24 | 23 | 1 | >24 | >24 | >9 < 23 |
| Lactobacillus acidophilus | 24 | 24 | >24 | >24 | >24 | 5 | >24 | >24 | >24 |
| Lactobacillus acidophilus | 24 | 24 | >24 | >24 | >24 | 23 | >24 | >24 | >24 |

### EXAMPLE #9

### Results of A pilot Clinical Study of Two Buffered Miconazole Vaginal Formulations

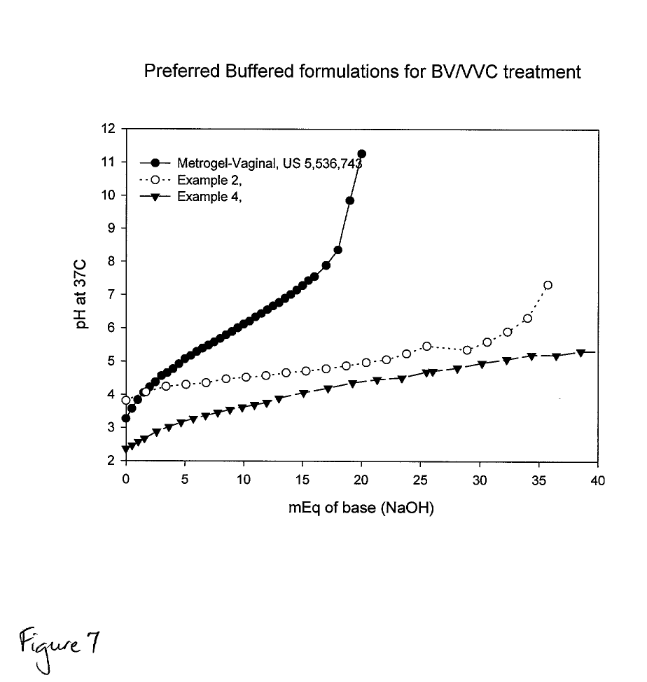

A Phase II in vivo pilot study was conducted to evaluate the therapeutic efficacy of two preferred buffered (4%) miconazole nitrate formulations (prototypes #1 and #2) compared with MetroGel-Vaginal® gel for the treatment of bacterial vaginosis (BV) when administered intravaginally. All products were administered daily for 5 days. The efficacy parameters for this pilot study were therapeutic cure rate (combined clinical and microbiological cure), clinical cure (relief of signs and symptoms) and microbiogical cure (Nugent score of 3 or less). Therapeutic, clinical and microbiological cure rates at return office visit scheduled 21-30 days after the initial dose of treatment were similar for miconazole nitrate buffered cream and Metrogel® vaginal. Therefore the buffered miconazole cream product administered for five days appears to be effective in et treatment of bacterial vaginosis. Vulvovaginal adverse events were reported by 50-60% of miconazole-treated subjects and 21% of Metrogel-treated subjects. Most adverse events were mild or moderate in intensity. FIG. 7 shows preferred buffered formulations for BV/VVC treatment.

| Ingredient | % w/w |
|---|---|
| Stearyl Alcohol | 8.5 |
| Cetyl Alcohol | 3 |
| Polysorbate 60 | 3 |
| Isopropyl Myristate | 1 |
| Propylene Glycol | 20 |
| Benzoic Acid | 0.2 |
| Potassium Hydroxide | 0.055 |
| Glucono Delta Lactone (GDL) | 1.8 |
| Miconazole Nitrate | 4 |
| Purified Water | 58.445 |

Prototype#2: Buffered Miconazole Nitrate Vaginal Gel

| Ingredient | % w/w |
|---|---|
| Carbomer 971 | 2 |
| Mineral Oil | 4.2 |
| Carbomer 974 | 1 |
| Distilled Monoglycerides | 1 |
| Sorbic Acid | 0.08 |
| Polyethylene Glycol 400 | 12.9 |
| Miconazole Nitrate | 4 |
| Purified Water | 74.82 |

What is claimed is:

1. A composition for simultaneously treating vulvovaginitis and vaginosis comprising:
a) A therapeutically effective amount of an antifungal agent; and
b) a buffering system capable of maintaining the pH of an infected area to which said composition is applied between about 2.5 and about 5.5.

2. A composition for simultaneously treating vulvovaginitis and vaginosis comprising:
a) an azole antifungal agent; and
b) a buffering system comprising a buffer selected from the group consisting of gluconodeltalactone, acetic acid, fumaric acid, lactic acid, citric acid, propionic acid, malic acid, succinic acid, gluconic acid, ascorbic acid and tartaric acid.

3. A composition for treating vulvovaginitis and vaginosis comprising:
a) a therapeutically effective amount of an azole antifungal agent;
b) a buffering system;
c) a pharmaceutically acceptable carrier.

4. An emulsion composition for treating vulvovaginitis and vaginosis comprising:
a) a therapeutically effective amount of an azole antifungal agent;
b) a buffering system comprising gluconodeltalactone;
c) a carbomer; and
d) a pharmaceutically acceptable carrier.

5. A gel composition for treating vulvovaginitis and vaginosis comprising:
a) a therapeutically effective amount of an azole antifungal agent;
b) a buffering system;
c) polyethylene glycol; and
d) a pharmaceutically acceptable carrier.

6. A dual-phase composition for treating vulvovaginitis and vaginosis comprising:
a) an oil phase comprising an azole antifungal agent and oil; and
b) a water phase comprising a buffering system and water; and
c) a pharmaceutically acceptable carrier.

7. A method for treating vulvovaginitis and vaginosis comprising administering to a mucous membrane a composition comprising an azole antifungal agent and a buffering system.

8. A composition according to claim 1 wherein said buffering system maintains the pH at from about 3 to about 5.

9. A composition according to claim 1 wherein said buffering system maintains the pH at from about 3 to about 4.5.

10. A method according to claim 7 wherein said mucous membrane is vaginal.

11. A method according to claim 7 wherein said mucous membrane is buccal.

12. A composition for the prophylaxis of vulvovaginitis and vaginosis comprising:
a) A therapeutically effective amount of an antifungal agent; and
b) a buffering system capable of maintaining the pH of an infected area to which said composition is applied between about 2.5 and about 5.5.

13. A composition for the prophylaxis of vulvovaginitis and vaginosis comprising:
a) an azole antifungal agent; and
b) a buffering system comprising a buffer selected from the group consisting of gluconodeltalactone, acetic acid, fumaric acid, lactic acid, citric acid, propionic acid, malic acid, succinic acid, gluconic acid, ascorbic acid and tartaric acid.

14. A composition for treating vulvovaginitis and vaginosis comprising:
a) a therapeutically effective amount of an azole antifungal agent;
b) a buffering system;
c) a pharmaceutically acceptable carrier.

15. An emulsion composition for the prophylaxis of vulvovaginitis and vaginosis comprising:
a) a therapeutically effective amount of an azole antifungal agent;
b) a buffering system comprising gluconodeltalactone;
c) a carbomer; and
d) a pharmaceutically acceptable carrier.

16. A gel composition for the prophylaxis of vulvovaginitis and vaginosis comprising:
a) a therapeutically effective amount of an azole antifungal agent;
b) a buffering system;
c) polyethylene glycol; and
d) a pharmaceutically acceptable carrier.

17. A method of preventing vulvovaginitis and vaginosis comprising applying a prophylactically effective amount of a compositions according to claim 1 to the vulva or vagina.

18. A method according to claim 17 which comprises applying to the vagina or vulva a composition according to claim 5.

19. A kit for treating vulvovaginitis and vaginosis comprising a composition according to claim 1 and a composition for soothing irritated skin.
